(19) United States
(12) Patent Application Publication
(10) Pub. No.: US 2002/0183288 A1
(43) Pub. Date: Dec. 5, 2002

(54) Method for treating and preventing hyperparathyroidism

(75) Inventors: Richard B. Mazess, Madison, WI (US); Stephen A. Strugnell, Madison, WI (US); Joyce C. Knutson, Madison, WI (US)

Correspondence Address: MICHAEL BEST & FRIEDRICH, LLP, ONE SOUTH PINCKNEY STREET, P O BOX 1806, MADISON, WI, 53701

(73) Assignee: Bone Care International, Inc., Middleton, WI

(21) Appl. No.: 10/127,005

(22) Filed: Apr. 19, 2002

Related U.S. Application Data
(63) Continuation-in-part of application No. 09/501,093, filed on Feb. 9, 2000, now Pat. No. 6,376,479.
Application No. 09/501,093 is a continuation-in-part of application No. 09/086,969, filed on May 29, 1998, now Pat. No. 6,242,434.
Application No. 09/086,969 is a continuation-in-part of application No. 08/907,659, filed on Aug. 8, 1997, now Pat. No. 5,869,473.
(63) Continuation-in-part of application No. 08/907,660, filed on Aug. 8, 1997, now abandoned.
Application No. 08/907,660 is a division of application No. 08/798,958, filed on Feb. 11, 1997, now Pat. No. 5,707,980.
Application No. 08/798,958 is a continuation of application No. 08/415,488, filed on Apr. 3, 1995, now Pat. No. 5,602,116.

(51) Int. Cl. A61K 31/59

(52) U.S. Cl. 514/167

(57) ABSTRACT

This invention relates to a method for treating or preventing hyperthyroidism associated with aging and/or with Aging-Related Vitamin D Deficiency (ARVDD) syndrome by administering a sufficient amount of an active vitamin D compound utilizing a variety of effective treatment protocols. The invention further relates to treating or preventing one or more of the following conditions, e.g., (1) primary vitamin D deficiency, (2) 1,25-(OH)₂D₃ deficiency, and (3) 1,25-(OH)₂D₃ resistance included within the syndrome of ARVDD.

### CROSS-REFERENCE TO RELATED APPLICATIONS

This application is a continuation-in-part of U.S. patent application Ser. No. 09/501,093 filed Feb. 9, 2000 which is a continuation-in-part of U.S. patent application Ser. No. 09/086,969, filed May 29, 1998 which is a continuation-in-part of U.S. patent application Ser. No. 08/907,659 filed Aug. 8, 1997, now U.S. Pat. No. 5,869,473, and this application is a continuation-in-part of U.S. patent application Ser. No. 08/907,660 filed Aug. 8, 1997 which is a divisional of U.S. patent application Ser. No. 08/798,958, filed Feb. 11, 1997, now U.S. Pat. No. 5,707,980, which is a continuation of U.S. patent application Ser. No. 08/415,488, filed Apr. 3, 1995, now U.S. Pat. No. 5,602,116.

### STATEMENT REGARDING FEDERALLY SPONSORED RESEARCH OR DEVELOPMENT

Not Applicable.

### BACKGROUND OF THE INVENTION

This invention relates to a method for treating or preventing hyperparathyroidism associated with aging by administering a sufficient amount of an active vitamin D compound utilizing a variety of effective treatment protocols. The method also relates to treating or preventing hyperparathyroidism associated with Aging-Related Vitamin D Deficiency (ARVDD) syndrome. Included within the syndrome of ARVDD are one or more of the following conditions, (1) primary vitamin D deficiency, (2) 1,25-(OH)₂D₃ deficiency, and (3) 1,25-(OH)₂D₃ resistance due to decreased responsiveness of target organs. ARVDD typically produces elevated blood parathyroid hormone levels, i.e., hyperparathyroidism. The invention is also a method of treating one or more of the conditions included within the syndrome of ARVDD.

In general, there are a number of physiological changes that occur with aging. One such change is the serum parathyroid hormone (PTH) level which has been found to increase with age. The cause of this increase in PTH (which has been found to be as high as 50% greater at age 80 as compared to a basal level seen at age 30) is not entirely clear. It has been suggested that some form of vitamin D deficiency is likely implicated. [See, e.g., Lau, K. -H. W. and Baylink, D. J., Calcif. Tissue Int. 65:295-306 (1999); Pattanaungkul, S., et al., J. Clinical Endocrinol. & Metab. 85:11 4023-4027 (2000)]. Another change associated with aging is the decline in muscle strength. It has also been suggested that some form of vitamin D deficiency may be implicated. [Grady, D. et al., J. Clin. Endocrinol. & Metab. 73:1111-1117 (1991); Bischoff, H. A. et al., Arch. Phys. Med. Rehabil. 80:54-58 (1999); Theiler, R. et al., Arch. Phys. Med. Rehabil. 80:485-489 (1999); Bischoff, H. A. et al., Histochem. J. 33:19-24 (2001); Glerup, H. “Investigations on the role of vitamin D in muscle function,” Ph.D. Thesis, Aarhus Bone and Mineral Research Group, University of Asrhus, Denmark (1999); Gulbrandsen, C. E. and Moss, R. L., U.S. Pat. No. 5,350,745, issued Sep. 27, 1994)]

Within the syndrome of ARVDD, there appear to be at least three different subtypes of vitamin D deficiency, each of which can occur with aging, and is characterized by an inadequate amount or insufficient biological action of 1,25-hydroxyvitamin D₃. These subtypes of vitamin D deficiency include (1) primary vitamin D deficiency, i.e., inadequate supplies of the precursors, vitamin D and/or 25-hydroxyvitamin D₃ leading to insufficient production of 1,25-dihydroxyvitamin D₃; (2) 1,25-dihydroxyvitamin D₃ deficiency, i.e., reduced abilities of the kidney to produce 1,25-dihydroxyvitamin D₃; and (3) 1,25-dihydroxyvitamin D₃ resistance, i.e., reduced responsiveness of target organs to 1,25-dihydroxyvitamin D₃ actions. [See, e.g., Lau, K. -H. W. and Baylink, D. J., Calcif. Tissue Int. 65:295-306 (1999); Pattanaungkul, S et al., J. Clinical Endocrinol. & Metab. 85:11 4023-4027 (2000)].

Primary vitamin D deficiency is caused by an inadequate supply of precursors, i.e., vitamin D and/or 25-hydroxyvitamin D₃, resulting in low serum levels of 1,25-dihydroxyvitamin D₃. Vitamin D is supplied to the human body via photosynthesis in the skin as a response to the UV-B radiation of sunlight or it is obtained through dietary sources. Inadequate sunlight exposure, which is regularly seen in countries of northern latitudes [Heikinehimo, R et al., (1992) Calcif Tissue Int 51:105-110], or insufficient nutritional vitamin D intake, which is a common problem of the elderly [Toss, G et al., Acta Med Scand 208:87-89 (1980)], are frequent causes of primary vitamin D deficiency. The photosynthesized or absorbed vitamin D undergoes 25-hydrpxylation to produce 25-dihydroxyvitamin D₃ in the liver. This hepatic hydroxylation is unregulated and solely substrate dependent. After production in the liver, 25-dihydroxyvitamin D₃ is converted to the physiologically active 1,25-dihydroxyvitamin D₃ in the kidney by the renal 25-hydroxyvitamin D-1α-hydroxylase. An inadequate vitamin D supply can lead to reduced levels of 25-hydroxyvitamin D₃, which then limits 1,25-dihydroxyvitamin D₃ production, resulting in low 1,25-dihydroxyvitamin D₃ levels, i.e., vitamin D deficiency [Ooms, ME et al., J. Bone Miner Res. 10:1177-1184 (1995)]. Thus, a low serum 25-dihydroxyvitamin D₃ level is a frequently used diagnostic hallmark for primary vitamin D deficiency. Primary vitamin D deficiency is not merely a biochemical abnormality; it is also associated with secondary hyperparathyroidism, increased bone turnover, bone loss, osteoporosis [Id.; Khaw, K. T. et al., Br. Med. J. 305:273-277 (1992)], and an increased risk of fractures [Eastell, R and Roggs, B. L., “Vitamin D and osteoporosis”, Vitamin D, Feldman D, Glorieux F H, Pike J W (eds) Academic Press, San Diego, Calif. pp. 695-711 (1997); Chapuy, M. C. and Meunier, P. J., “Vitamin D insufficiency in adults and the elderly”, Vitamin D, Feldman D, Glorieux F H, Pike J W (eds) Academic Press, San Diego, Calif. pp. 679-693 (1997); Lau, K. -H. W. and Baylink, D. J., supra].

1,25-(OH)₂D₃ deficiency, unlike primary vitamin D deficiency, is not caused by a limitation of precursors, e.g., vitamin D and/or 25-dihydroxyvitamin D₃, but rather by a defect in the synthesis of 1,25-dihydroxyvitamin D₃. 1,25-dihydroxyvitamin D₃ deficiency causes a decrease in intestinal calcium absorption, increased serum PTH, increased bone resorption, bone loss, and osteoporosis. The pathogenesis of 1,25-dihydroxyvitamin D₃ deficiency is related to an impaired ability of the kidney to synthesize adequate amounts of 1,25-dihydroxyvitamin D₃ rather than an inadequate supply of the substrate 25-hydroxyvitamin D₃. 1,25-dihydroxyvitamin D₃ deficiency is common in patients with renal insufficiency, renal failure, or other renal diseases. Thus, low serum levels of 25-hydroxyvitamin D₃ are not characteristic of 1,25-dihydroxyvitamin D₃ deficiency. Low serum 1,25-dihydroxyvitamin D₃ levels, normal serum 25-hydroxyvitamin D₃ levels, calcium malabsorption, secondary hyperparathyroidism, increased bone turnover, and bone loss are diagnostic indicia of 1,25-dihydroxyvitamin D₃ deficiency. [See, Lau, K. -H. W. and Baylink, D. J., supra].

There is also evidence that 1,25(OH)₂D₃ resistance is present in the elderly. The aging-associated decline in functions of various tissues and organs in the elderly can produce resistance of target organs to 1,25-dihydroxyvitamin D₃, leading to reduced biological actions of the hormone. Higher levels of 1,25-dihydroxyvitamin D₃ are needed in patients with the 1,25-dihydroxyvitamin D₃ resistance to achieve the same levels of 1,25-dihydroxyvitamin D₃ hormonal actions as those seen in normal individuals. “Normal” 1,25-dihydroxyvitamin D₃ levels, which are adequate for normal subjects, are insufficient to meet the physiological needs of resistant patients. However, unlike 1,25-dihydroxyvitamin D₃ deficiency, which has a lower serum 1,25-dihydroxyvitamin D₃ level, 1,25-dihydroxyvitamin D₃ resistance would be expected to show normal or slightly elevated (due to feedback regulation) serum 1,25-dihydroxyvitamin D₃ levels. Yet, in spite of elevated levels of serum 1,25-dihydroxyvitamin D₃, these patients would exhibit all the metabolic features of vitamin D deficiency; i.e., reduced intestinal calcium absorption, secondary hyperparathyroidism, increased bone turnover, and bone loss. Consequently, a typical patient with 1,25-dihydroxyvitamin D₃ resistance would have normal serum 25-hydroxyvitamin D₃ levels, and normal or slightly elevated 1,25-dihydroxyvitamin D₃ serum levels, but at the same time would exhibit reduced intestinal calcium absorption, secondary hyperparathyroidism, increased bone resorption, bone loss, and osteoporosis. [See, Lau, K. -H. W. and Baylink, D. J., supra].

As noted herein above, serum PTH levels increase with age, and secondary hyperparathyroidism has been associated with aging. The disease of hyperparathyroidism is a generalized disorder resulting from excessive secretion of parathyroid hormone by one or more parathyroid glands. The disease is characterized by elevated blood parathyroid hormone levels and parathyroid glandular enlargement.

Hyperparathyroidism is subcategorized into primary, secondary and tertiary hyperparathyroidism. In primary hyperparathyroidism, the growth of the parathyroid glands is autonomous in nature, is usually due to tumors, e.g., parathyroid adenomas, and is presumably irreversible. Such adenomas typically do not exhibit vitamin D receptors and exhibit a resistivity to 1,25-dihydroxyvitamin D₃. In secondary hyperparathyroidism, associated, e.g., with 1,25-dihydroxyvitamin D₃ deficiency and/or resistance, the parathyroid gland hyperplasia is typically adaptive owing to resistance to the metabolic actions of the hormone, and is presumably reversible. Secondary hyperparathyroidism occurs in patients, e.g., with renal failure, osteomalacia, and intestinal malabsorption syndrome. Tertiary hyperparathyroidism is characterized by an autonomous proliferation state of the parathyroid glands with biological hyperfunction. Tertiary hyperparathyroidism can occur in patients with secondary hyperparathyroidism, wherein the reversible hyperplasia associated with secondary hyperparathyroidism converts to an irreversible growth defect, the enlarged tissue having vitamin D receptors. In all forms of hyperparathyroidism, bone abnormalities, e.g., the loss of bone mass or decreased mineral content, are common and renal damage is possible. Hyperparathyroidism is thus also characterized by abnormal calcium, phosphorus and bone metabolism.

Historically, it has long been known that vitamin D plays a critical role regulating calcium metabolism. The discovery of the active forms of vitamin D in the 1970's [Holick, M. F. et al., Proc. Natl. Acad. Sci. USA 68, 803-804 (1971); Jones, G. et al., Biochemistry 14, 1250-1256 (1975)] and active vitamin D analogues [Holick, M. F. et al., Science 180, 190, 191 (1973); Lam, H. Y. et al., Science 186, 1038-1040 (1974)], caused much excitement and speculation about the usefulness of these compounds in the treatment of bone depletive disorders.

Animal and early clinical studies examining the effects of these active vitamin D compounds suggested that such agents would be useful in restoring calcium balance. However, the best indicator of the efficacy of vitamin D compounds to prevent or treat depletive bone disorders is bone itself (or, in the case of renal osteodystrophy, serum levels of parathyroid hormone (PTH)) rather than calcium absorption or calcium balance. Certain clinical studies with 1α,25-dihydroxyvitamin D₃, and 1α-hydroxyvitamin D₃ indicate that the ability of these agents to restore lost bone mass or bone mineral content is dose-related. [See, Ott, S. M. and Chesnut, C. H., Annals of Int. Med.; 110:267-274 (1989); Gallagher, J. C. et al., Annals of Int. Med.; 113:649-655 (1990); Aloia, J. et al., Amer. J. Med. 84:401-08 (1988); and Shiraki, M. et al., Endocrinol. Japan 32, 305-315 (1985)].

These clinical studies also indicate that at the dosage ranges required for these agents to be truly effective, toxicity in the form of hypercalcemia and hypercalciuria becomes a major problem. Attempts to increase the amount of 1α,25-dihydroxyvitamin D₃ above 0.5 μg/day have frequently resulted in toxicity. At dosage levels below 0.5 μg/day, clinically significant effects on bone are rarely observed. [See, Jensen, G. F. et al., Clin. Endocrinol. 16, 515-524 (1982); Christiansen, C. et al., Eur. J. Clin. Invest. 11, 305-309 (1981)]. Doses of 2 μg/day of 1α-hydroxyvitamin D₃ (1α-(OH)D₃) were found to have efficacy in increasing bone mass in patients exhibiting senile osteoporosis [Sorensen, O. H. et al., Clin. Endocrinol. 7, 169S-175S (1977)]. Data from clinical studies in Japan, a population that has low calcium intake, indicate that efficacy is found with 1α-hydroxyvitamin D₃ when administered at 1 μg/day [Shiraki, M. et al., Endocrinol. Japan. 32:305-315 (1985); Orimo, H. et al., Bone and Mineral 3, 47-52 (1987)]. However, at 2 μg/day, toxicity with 1α-hydroxyvitamin D₃ occurs in approximately 67% of the patients, and at 1 μg/day this percentage is approximately 20%.

Thus, due to their toxicity, 1-hydroxylated vitamin D₃ compounds can only be administered at dosages that are, at best, modestly beneficial in preventing or treating loss of bone or bone mineral content. Indeed, Aloia et al., recommend that alternative routes of administration be sought that might avoid the toxicity problems and allow higher dosage levels to be achieved. [Aloia, J. et al., Amer. J. Med. 84:401-408 (1988)]. Despite reported toxicities of 1α-hydroxyvitamin D₃ and 1α,25-dihydroxyvitamin D₃, these two compounds remain the drugs of choice for treatment of many bone depletive diseases.

Both 1α-hydroxyvitamin D₃ and 1α,25-dihydroxyvitamin D₃ have been studied and are clinically used in certain countries in Asia and Europe to treat osteoporosis [Gillespie, W. J., et al., Abstract, The Cochrane Library, issue 2, 2001; DeChant, K. L. and Goa, K. L., Drugs & Aging, 5(4):300-317 (1994); Ikeda, K and Ogata, E., Mechanisms of Aging & Development 116:103-111 (2000); Tanizawa, T., Osteoporos. Int. 9:163-170 (1999); Civitelli, R., Calcif. Tissue 57:409-414 (1995); Parfitt, A. M., Drugs 36:513-520 (1988); Thompson, S. P. et al., Brit. Edit. Soc. Bone Joint Surgery, 72:1053-1056 (1990); Sairanen, S. et al., Calcif. Tissue Int. 67:122-127 (2000); Haas, H. G., Horm. Metab. Res. 11:168-171 (1979); Tilyard, M. W. et al., New England J. Med. 326:357-362 (1992); Aloia, J. F. et al., Am. J. Med. 84:401-408 (1988); Avioli, L., Calcif. Tissue Int. 65:2392-294 (1999); Orimi, H. et al., Calcif. Tissue Int. 54:370-376 (1994); Sorensen, O. H. et al., Clinical Endocrinol. 7 (Suppl.): 169S-175S (1997)] Some studies suggest that active vitamin D, such as 1α-hydroxyvitamin D₃ and 1α,25-dihydroxyvitamin D₃, appears to be more effective than precursors, e.g., vitamin D, in treating, e.g., osteoporosis. These drugs appear to be most effective in those patients that have defective calcium absorption, e.g., in osteoporosis. Active vitamin D also appears to be more effective in treating 1α,25-dihydroxyvitamin D₃ resistance in target organs, decline in responsiveness to PTH inducement of 1α,25-dihydroxyvitamin D₃ synthesis, and lower production of 1α,25-dihydroxyvitamin D₃ especially with aging. [Zerwekh, J. E. et al., J. Clin. Endocrinol. Metab. 56:410-413 (1983); Nordin, B. E. C. et al., Calcif. Tissue Int. 65:307-310 (1999); Morris, H. A. et al., Calcif. Tissue Int. 49:240-243 (1991); Shiraishi, A. et al., Calcif. Tissue Int.65:311-316 (1999); Silverberg, S. J. et al., New England J. Med . 320(5):277-281 (1989); Francis, R. M., Calcif. Tissue Int. 60:111-114 (1997); Francis, R. M. et al., Osteoporosis Int. 6:284-290 (1996); Theiler, R. et al., Int. J. Vit. Nur. Res. 68:36-41 (1998)]

Also, as noted above, secondary hyperparathyroidism is a significant clinical problem associated with renal insufficiency and intestinal malabsorption syndromes, and has also been associated with aging as described herein above. As to renal failure, in the United States, end stage renal disease afflicts approximately 300,000 individuals. In this disease, there is a progressive loss of cells of the proximal nephrons, the primary site for the synthesis of the vitamin D hormones (collectively “1α,25-(OH)₂D”) from 25-hydroxyvitamin D₃ and 25-hydroxyvitamin D₂. In addition, the loss of functioning nephrons leads to retention of excess phosphorus which reduces the activity of the renal 25-hydroxyvitamin D-1α-hydroxylase, the enzyme which catalyzes the reaction to produce the D hormones. These two events account for the low serum levels of 1α,25-(OH)₂D commonly found in patients with mild to moderate end stage renal disease.

Reduced serum levels of 1α,25-(OH)₂D cause increased, and ultimately excessive, secretion of PTH by direct and indirect mechanisms. The resulting hyperparathyroidism leads to markedly increased bone turnover and its sequela of renal osteodystrophy, which may include a variety of other diseases, such as, osteitis fibrosa cystica, osteomalacia, osteoporosis, extraskeletal calcification and related disorders, e.g., bone pain, periarticular inflammation and Mockerberg's sclerosis. Reduced serum levels of 1α,25-(OH)₂D also can cause muscle weakness and growth retardation with skeletal deformities (most often seen in pediatric patients).

Previous clinical studies of hormonally active vitamin D drugs in end stage renal disease patients, i.e., the treatment of secondary hyperthyroidism, have focused on compounds derived from vitamin D₃. 1α,25-(OH)₂D₃ and 1α-(OH)D₃ are the major approved forms of 1α-hydroxylated vitamin D for treatment or prevention, although these drugs are not currently approved in all major pharmaceutical markets. Use of 1α,25-(OH)₂D₃ and 1α-(OH)D₃ as replacement therapy seeks to treat or prevent renal osteodystrophy by treating or preventing hyperparathyroidism in end stage renal disease patients. As noted above, 1α,25-(OH)₂D₃ often causes toxic side effects (hypercalcemia and hyperphosphatemia) at dosages above 0.5 μg, especially when concomitantly administered phosphate binders, such as calcium compounds, are used to control serum phosphorus. The minimum effective dose for preventing hyperparathyroidism is in the range of 0.25 to 0.50 μg/day; most patients respond to oral treatment doses of 0.5 to 1.0 μg/day or intravenous doses between 0.5 and 3.0 μg three times per week. As described above, the other commonly used vitamin D drug is 1α-(OH)D₃ which often causes toxic effects at dosages over 1.0 μg/day, especially when used with phosphate binders. The minimum effective dosage for preventing hyperparathyroidism is in the range of 0.25 to 1.0 μg/day, and most patients require treatment dosages of 1.0 μg/day or more. When either drug, 1α,25-(OH)₂D₃ or 1α-(OH)D₃, is administered in higher dosages, both efficacy and toxicity are found to increase. Thus, the hormonally active vitamin D₃ compounds are limited in their therapeutic usefulness due to their inherent toxicities.

Attempts to reduce the toxic side effects of active vitamin D₃, in the renal failure setting have included administration of a low calcium dialysate with an ionized calcium concentration of 1.25 mM. However, it has been found that use of the low calcium dialysate has lead to higher serum PTH and phosphorus levels in patients who do not receive increased doses of oral calcium supplements and phosphate binders. When the dosages of calcium supplements and phosphate binders are increased, serum levels of phosphorus can be controlled, but the incidence of hypercalcemia rises markedly. Thus, there are many problems associated with the use of current vitamin D therapies for secondary hyperparathyroidism.

As to secondary hyperparathyroidism associated with aging, it has been suggested that treatment with 1α-OH-D₃ is advantageous over vitamin D. [Shiraishi, A et al., Calcif. Tissue Int. 65:292-294 (1999)]. However, although active forms of vitamin D₃ may have increased efficacy over precursors, their inherent toxicities still limit extensive therapeutic use.

Notwithstanding these known problems with use of the hormonally active vitamin D₃ for hyperparathyroidism, there is a need for vitamin D compounds, derivatives or analogs, and treatment protocols that have low inherent toxicity.

### BRIEF DESCRIPTION OF THE INVENTION

In one aspect, the present invention provides a method of treating, i.e., ameliorating or preventing, hyperparathyroidism associated with aging. The method includes administering to a subject in need thereof an amount of an active vitamin D compound sufficient to lower elevated or maintain lowered blood parathyroid hormone (PTH) levels, i.e., sufficient to suppress parathyroid activity.

In another aspect, the invention provides a method of treating or preventing hyperparathyroidism associated with Aging-Related Vitamin D Deficiency (ARVDD) syndrome. The method includes administering an amount of an active vitamin D compound to a subject in need sufficient to lower elevated or maintain lowered blood parathyroid hormone levels. ARVDD includes one or more of primary vitamin D deficiency, 1,25-(OH)₂D₃ deficiency and 1,25-(OH)₂D₃ resistance. Thus, in a further aspect, the invention provides a method of treating or preventing one or more of the conditions associated with ARVDD.

The method further includes administration of the active vitamin D by a variety of effective treatment protocols. One such protocol includes intermittent or episodic high dose regimen of the active vitamin D compound. The active vitamin D compounds in accordance with the present invention have bioactivity equivalent to, but have lower toxicity than, conventional vitamin D therapies.

A fuller appreciation of the invention will be gained upon an examination of the following description, taken in conjunction with the appended claims.

### BRIEF DESCRIPTION OF THE DRAWINGS

Not Applicable.

### DESCRIPTION OF THE INVENTION

The present invention relates to ameliorating or preventing hyperparathyroidism associated with aging and/or associated with Aging-Related Vitamin D Deficiency (ARVDD) syndrome by administering an effective amount of an active vitamin D compound utilizing a variety of treatment protocols. ARVDD includes within the syndrome one or more of the following conditions, (1) primary vitamin D deficiency, (2) 1,25-(OH)₂D₃ deficiency, and (3) 1,25-(OH)₂D₃ resistance. An elevated blood parathyroid hormone level, i.e., hyperparathyroidism, is typically associated with aging and with one or more of the conditions within the syndrome of ARVDD. Accordingly, the present invention will now be described in detail with respect to such endeavors; however, those skilled in the art will appreciate that such a description of the invention is meant to be exemplary only and should not be viewed as limitative on the full scope thereof.

More specifically, the present invention relates to therapeutic methods for lowering elevated blood levels of parathyroid hormone (PTH) and/or maintaining lowered serum PTH levels associated with aging and/or ARVDD. The method is of value in ameliorating or preventing one or more of the conditions included within the syndrome of ARVDD by, e.g., minimizing vitamin D deficiency, increasing renal production of 1,25-(OH)₂D₃, and reducing 1,25-(OH)₂D₃ resistance in target organs. The method in accordance with the present invention has significantly less resultant hypercalcemia and hyperphosphatemia, especially in patients who use oral calcium as a phosphate binder to control serum phosphorus levels. Furthermore, the active vitamin D compounds when administered intermittently or episodically in a high dose regimen result in higher efficacy and reduced toxicity. These attributes are achieved through a novel method of treating patients suffering from hyperparathyroidism associated with aging and/or with one or more of the conditions associated with ARVDD.

As used herein, the term “Aging-Related Vitamin D Deficiency syndrome (ARVDD)” refers to one or more of the conditions of primary vitamin D deficiency, 1,25-(OH)₂D₃ deficiency and 1,25-(OH)₂D₃ resistance that can occur in the elderly. In addition to poor sunlight exposure and decreased vitamin D intake, other factors that probably contribute to this ARVDD syndrome include defective renal production of 1,25-(OH)₂D₃, and a progressive decrease in the number of the 1,25-(OH)₂D₃ receptor (VDR) complexes which can transduce its biological effects on the intestine and bone.

Also, as used herein, the term “hyperparathyroidism” refers to primary, secondary and/or tertiary hyperparathyroidism.

It has been found that when the analogs of formula (I), described hereinbelow, are administered to patients with elevated serum parathyroid hormone, PTH concentration is lowered with significantly less hypercalcemia and hyperphosphatemia than is observed after the same amount of activated vitamin D₃ administered in previously known formulations and dosing regimens. Thus, the compounds of formula (I) have an improved therapeutic index relative to active vitamin D₃ analogs administered using conventional protocols.

It has been shown that 1α-hydroxyvitamin D₂ (1α-(OH)D₂) has the same biopotency as 1α-hydroxyvitamin D₃ (1α-(OH)D₃) and 1α,25-dihydroxyvitamin D₃ (1α,25-(OH)₂D₃) but is much less toxic [see, U.S. Pat. No. 5,403,831 and U.S. Pat. No. 5,104,864]. Even dosages up to 10 μg/day of 1α-(OH)D₂ in women with postmenopausal osteoporosis (in both open label and double blind testing) exhibited only mild hypercalciuria (>300 mg/24 hrs), and marked hypercalcemia (>11.0 mg/dL) solely due to 1α-(OH)D₂ was not evident. Additionally, 1α-(OH)D₂ did not adversely affect kidney function, as determined by creatinine clearance and BUN; nor did it increase urinary excretion of hydroxyproline, indicating the absence of any stimulatory effect on bone resorption. Administration of 1α-(OH)D₂ to healthy adult males in dosages up to 8 μg/day has shown no hypercalcemia or other adverse effects.

Furthermore, it is known that vitamin D₃ must be hydroxylated in the C-1 and C-25 positions before it is activated, i.e., before it will produce a biological response. A similar metabolism appears to be required to activate other forms of vitamin D, e.g., vitamin D₂ and vitamin D₄. Therefore, as used herein, the term “activated vitamin D” or “active vitamin D” is intended to refer to a vitamin D compound or analog that has been hydroxylated in at least one of the C-1, C-24 or C-25 positions of the molecule (i.e., a hydroxy vitamin D) and either the compound itself, or one of its metabolites in the case of a prodrug, binds to the vitamin D receptor (VDR). For example, vitamin D “prodrugs” include compounds that are hydroxylated in the C-1 position. Such compounds undergo further hydroxylation in vivo and their metabolites bind the VDR.

Also, as used herein, the term “lower” as a modifier for alkyl, alkenyl, acyl, or cycloalkyl is meant to refer to a straight or branched, saturated or unsaturated hydrocarbon radical having 1 to 4 carbon atoms. Specific examples of such hydrocarbon radicals are methyl, ethyl, propyl, isopropyl, butyl, isobutyl, t-butyl, ethenyl, propenyl, butenyl, isobutenyl, isopropenyl, formyl, acetyl, propionyl, butyryl or cyclopropyl. The term “aromatic acyl” is meant to refer to an unsubstituted or substituted benzyl group.

As used herein, the term “hydrocarbon moiety” refers to a lower alkyl, a lower alkenyl, a lower acyl group or a lower cycloalkyl, i.e., a straight or branched, saturated or unsaturated C₁-C₄ hydrocarbon radial.

Further, the active vitamin D in accordance with the present invention may have an unsaturated side chain, e.g., there is suitably a double bond between C-22 and C-23, between C-25 and C-26 or between C-26 and C-27.

An active vitamin D of the present invention i.e., a hydroxyvitamin D, has the general formula described in formula (I):

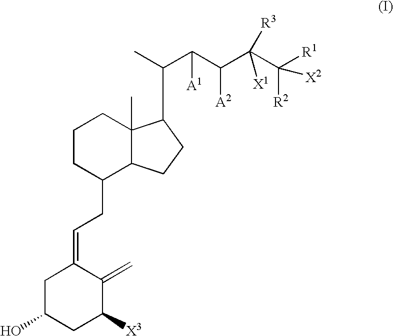

wherein A¹ and A² each are hydrogen or together represent a carbon-carbon bond, thus forming a double bond between C-22 and C-23; R¹ and R² are identical or different and are hydrogen, hydroxyl, lower alkyl, lower fluoroalkyl, O-lower alkyl, lower alkenyl, lower fluoroalkenyl, O-lower alkenyl, O-lower acyl, O-aromatic acyl, lower cycloalkyl with the proviso that both R¹ and R² cannot both be an alkenyl, or taken together with the carbon to which they are bonded, form a C₃-C₈ cyclocarbon ring; R³ is lower alkyl, lower alkenyl, lower fluoroalkyl, lower fluoroalkenyl, O-lower alkyl, O-lower alkenyl, O-lower acyl, O-aromatic acyl or lower cycloalkyl; X¹ is hydrogen or hydroxyl; X² is hydrogen or hydroxyl, or, may be taken with R¹ or R², to constitute a double bond; and X³ is hydrogen or hydroxyl provided that at least one of X¹, X² and X³ is hydroxyl.

Specific 1α-hydroxyvitamin D compounds in accordance with the present invention are characterized by the general formula (II):

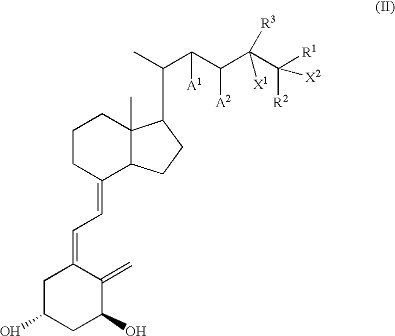

wherein A¹ and A² each are hydrogen or together represent a carbon-carbon bond, thus forming a double bond between C-22 and C-23; R¹ and R² are identical or different and are hydrogen, hydroxyl, lower alkyl, lower fluoroalkyl, O-lower alkyl, lower alkenyl, lower fluoroalkenyl, O-lower alkenyl, O-lower acyl, O-aromatic acyl, lower cycloalkyl with the proviso that both R¹ and R² cannot both be an alkenyl, or taken together with the carbon to which they are bonded, form a C₃-C₈ cyclocarbon ring; R³ is lower alkyl, lower alkenyl, lower fluoroalkyl, lower fluoroalkenyl, O-lower alkyl, O-lower alkenyl, O-lower acyl, O-aromatic acyl or lower cycloalkyl; X¹ is hydrogen or hydroxyl; and X² is hydrogen or hydroxyl, or, may be taken with R¹ or R², to constitute a double bond.

The active 1α-hydroxylated vitamin D analogs in accordance with the present invention wherein R¹, R², and R³ are all methyl groups and X² is hydrogen, have the general formula (III):

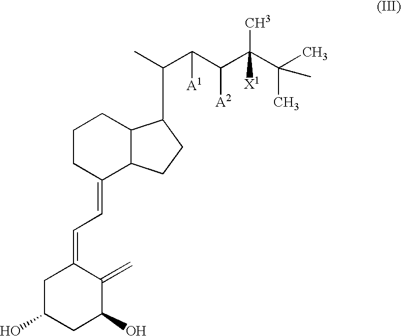

wherein A¹and A² are each either hydrogen, or together represent a carbon-carbon double bond; and X¹ is either hydrogen or hydroxyl.

Specific 24-hydroxyvitamin D compounds in accordance with the present invention are characterized by the general formula (IV):

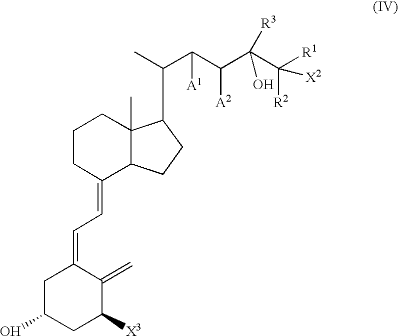

wherein A¹ and A² each are hydrogen or together represent a carbon-carbon bond, thus forming a double bond between C-22 and C-23; R¹ and R² are identical or different and are hydrogen, hydroxyl, lower alkyl, lower fluoroalkyl, O-lower alkyl, lower alkenyl, lower fluoroalkenyl, O-lower alkenyl, O-lower acyl, O-aromatic acyl, lower cycloalkyl with the proviso that both R¹ and R² cannot both be an alkenyl, or taken together with the carbon to which they are bonded, form a C₃-C₈ cyclocarbon ring; R³ is lower alkyl, lower alkenyl, lower fluoroalkyl, lower fluoroalkenyl, O-lower alkyl, O-lower alkenyl, O-lower acyl, O-aromatic acyl or lower cycloalkyl; X³ is hydrogen or hydroxyl, and X² is hydrogen or hydroxyl, or, may be taken with R¹ or R², to constitute a double bond.

Such compounds in accordance with formulas I-IV include generally 24-hydroxyvitamin D compounds, 25-hydroxyvitamin D compounds and 1α-hydroxyvitamin D compounds. Specific examples of such compounds of formulas (I)-(IV) include, without limitation, 1α,24-dihydroxyvitamin D₂, 1α,24-dihydroxyvitamin D₄, 1α,25-dihydroxyvitamin D₄, 1α,25-dihydroxyvitamin D₂, 1α,24,25-trihydroxyvitamin D₂, 1α,25-dihydroxyvitamin D₃, 1α,24,25-trihydroxyvitamin D₃, and also include such pro-drugs or pro-hormones as la-hydroxyvitamin D₂, 1α-hydroxyvitamin D₄, 24-hydroxyvitamin D₂, 24-hydroxyvitamin D₄, 25-hydroxyvitamin D₂, and 25-hydroxyvitamin D₄.

The compounds in accordance with the present invention are typically hypocalcemic compared to the natural D hormone, 1α,25-dihydroxyvitamin D₃. “Hypocalcemic” is meant to refer to an active vitamin D compound that has reduced calcemic activity compared to that of the natural vitamin D hormone, 1α,25-dihydroxyvitamin D₃; in other words, a calcemic index less than that of 1α,25-dihyroxyvitamin D₃. The calcemic activity of these compounds typically ranges from 0.001 to 0.5 that of 1α,25-dihydroxyvitamin D₃. “Calcemic index” is a relative measure of the ability of a drug to generate a calcemic response, the calcemic activity of 1α,25-dihydroxyvitamin D₃ being designated as 1. Such hypocalcemia vitamin D compounds provide reduced risk of hypercalcemia even when administered in high doses.

Further, for compounds of formulas (I)-(IV) that have a chiral center, such as at the C-24 position, it is understood that both epimers (e.g., R and S) and the epimeric mixture are within the scope of the present invention. Where certain epimeric forms are preferred, the preferred form is substantially free of its other epimeric form, e.g., 1α,24(S)-dihydroxyvitamin D₂ is preferably substantially free of its (R) epimer, and 1α,24(R)-dihydroxy vitamin D₄ is preferred substantially free of its (S) epimer.

The vitamin D analogs of formulas (I)-(IV) are useful as active compounds in pharmaceutical compositions. The active vitamin D compounds of the present invention include vitamin D compounds having a hydroxy group substituted in at least one of the C₁, C₂₄ or C₂₅ positions of the molecule, i.e., a hydroxy vitamin D. The analogs of formula (III) are of especial value as they are substantially less toxic than their vitamin D₃ counterparts when administered by conventional protocols to patients experiencing hyperparathyroidism. For example, in patients using oral calcium as a phosphate binder, e.g., calcium carbonate or calcium acetate, administration of the analogs of formula (III) at dosage levels higher than possible with the vitamin D₃ compounds provides greater efficacy than heretofore possible in treating hyperparathyroidism.

Effective amounts of active vitamin D compounds in accordance with the present invention may be administered on a daily or episodic basis. Dosages may be from 1 μg to 150 μg per week given daily or 10 μg/dose or greater up to 200 μg/dose or greater, given episodically or intermittently.

The method in accordance with the present invention also includes use of active vitamin D compounds, and of particular value, hypocalcemic active vitamin D compounds, especially compounds of vitamins D₂, D₃ and D₄, in high dosage form, administered on an intermittent or episodic basis, to treat hyperparathyroidism associated with aging and inhibit symptoms associated with ARVDD syndrome. The active vitamin D compounds given in episodic or intermittent high dose may also be co-administered with other therapeutic agents (as described in detail below). Administration of the active vitamin D may be prior to, simultaneous with, or after administration of the other therapeutic agents.

With the episodic or intermittent dosing protocol in accordance with the present, high dose amounts administered to patients having ARVDD may even include 1α,25-dihydroxyvitamin D₃ (calcitriol), or 1α-(OH)-D₃ (alphacalcidol). By “high dose” is meant a dose of 10 μg or more, e.g., 20 μg to 100 μg or more, e.g. 300 μg. In other terms, a “high dose” is one that produces higher than normal physiologic levels of vitamin D in vivo, or is sufficient in a single dose to upregulate vitamin D receptors on cells expressing these receptors. The intermittent dosing regimen is suitably between once per week to once every 12 weeks, e.g., once every 3 weeks. As a function of body weight, the effective dose ranges from about 0.2 μg to about 4.5 μg per kilogram of body weight of the patient.

The episodic protocol or dosage regimen in accordance with the present invention provides an improved therapeutic index for active forms of vitamin D analogues compared to administration via conventional regimens. The episodic dosing is also cost effective, as less active agent is needed.

It is further believed that the intermittent high dose regimen can be used to effect any therapeutic effect that is attributable to active vitamin D., e.g., reduction of loss of bone mass, etc. The value of the intermittent dosing is that upregulation of vitamin D receptors occurs with a single dose without the side effects of hypercalcemia and hypercalciuria that occur with recurrent daily dosing.

The episodic dose can be a single dose or, optionally, divided into 2-4 subdoses which, if desired, can be given, e.g., twenty minutes to an hour apart until the total dose is given. The compounds in accordance with the present invention are administered in an amount that raises serum vitamin D levels to a supraphysiological level for a sufficient period of time to alleviate, e.g., 1,25-(OH)₂D₃ deficiency and/or resistance without causing hypercalcemia or with substantially reduced the risk of hypercalcemia. The properties of the hypocalcemic vitamin D compounds in accordance with the present invention are particularly beneficial in permitting such supraphysiologic levels.

Generally, the pharmacologically active compounds of the present invention can be processed in accordance with conventional methods of pharmacy to produce medicinal agents for administration to patients, e.g., mammals including humans. For example, the active vitamin D compounds of the present invention can be formulated in pharmaceutical compositions in a conventional manner using one or more conventional excipients, which do not deleteriously react with the active compounds, e.g., pharmaceutically acceptable carrier substances suitable for enteral administration (e.g., oral), parenteral, topical, buccal or rectal application, or by administration by inhalation or insufflation (e.g., either through the mouth or the nose)

Generally, acceptable carriers for pharmaceutical formulation include, but are not limited to, water, salt solutions, alcohols, gum arabic, vegetable oils (e.g., almond oil, corn oil, cottonseed oil, peanut oil, olive oil, coconut oil), mineral oil, fish liver oils, oily esters such as Polysorbate 80, polyethylene glycols, gelatin, carbohydrates (e.g., lactose, amylose or starch), magnesium stearate, talc, silicic acid, viscous paraffin, fatty acid monoglycerides and diglycerides, pentaerythritol fatty acid esters, hydroxy methylcellulose, polyvinylpyrrolidone, etc.

Of particular interest is the parenteral, e.g., injectable, dosage form. Using the parenteral route of administration allows for bypass of the first pass of active vitamin D compound through the intestine, thus avoiding stimulation of intestinal calcium absorption, and further, reduces the risk of esophageal irritation which may be associated with high dose oral administration. Because an injectable route of administration is typically done by a health care professional, the dosing can be more effectively controlled as to precise amount and timing. Parenteral administration suitably includes subcutaneous, intramuscular, or intravenous injection, nasopharyngeal or mucosal absorption, or transdermal absorption.

Injectable compositions may take such forms as sterile suspensions, solutions, or emulsions in oily vehicles (such as coconut oil, cottonseed oil, sesame oil, peanut oil or soybean oil) or aqueous vehicles, and may contain various formulating agents. Alternatively, the active ingredient may be in powder (lyophilized or non-lyophilized) form for reconstitution at the time of delivery with a suitable vehicle, such as sterile water.

In injectable compositions, the carrier is typically sterile and pyrogen-free, e.g., water, saline, aqueous propylene glycol, or another injectable liquid, e.g., peanut oil for intramuscular injections. Also, various buffering agents, preservatives, suspending, stabilizing or dispensing agents, surface-active agents and the like can be included. Aqueous solutions may be suitably buffered, if necessary, and the liquid diluent first rendered isotonic with sufficient saline or glucose. Aqueous solutions are especially suitable for intravenous, intramuscular, subcutaneous and intraperitoneal injection purposes. In this connection, the sterile aqueous media employed are all readily obtainable by standard techniques well known to those skilled in the art. The oily solutions are especially suitable for intra-articular, intramuscular and subcutaneous injection purposes. The preparation of all these solutions under sterile conditions is readily accomplished by standard pharmaceutical techniques well known to those skilled in the art.

The compounds in accordance with the present invention formulated for parenteral administration by injection may be administered by bolus injection or continuous infusion. Formulations for injection may be conveniently presented in unit dosage form, e.g., in ampoules or in multi-dose containers, with an added preservative.

In addition to the formulations described previously, the compounds may also be formulated as a depot preparation. Such long acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, e.g., a sparingly soluble salt.

For enteral application, particularly suitable are tablets, dragees, liquids, drops, suppositories, lozenges, powders, or capsules. Syrup, elixir, or the like can be used if a sweetened vehicle is desired. For oral administration, the pharmaceutical compositions may take the form of, for example, tablets or capsules, e.g., soft or hard gel capsules, prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). Tablets may be coated by methods well known in the art.

Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may also be suitably formulated to give controlled release of the active compound. Many controlled release systems are known in the art.

For buccal administration, the compositions may take the form of tablets, lozenges or absorption wafers formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of e.g. gelatin, for use in an inhaler or insufflator may be formulated containing a powder mix of the active compound and a suitable powder base such as lactose or starch.

The compounds may also be formulated in rectal or vaginal compositions, such as suppositories containing conventional suppository bases or retention enemas. These compositions can be prepared by mixing the active ingredient with a suitable non-irritating excipient which is solid at room temperature (for example, 10° C. to 32° C.) but liquid at the rectal temperature, and will melt in the rectum or vagina to release the active ingredient. Such materials are polyethylene glycols, cocoa butter, other glycerides and wax. To prolong storage life, the compositions advantageously may include an antioxidant such as ascorbic acid, butylated hydroxyanisole or hydroquinone.

The compositions may, if desired, be presented in a pack or dispenser device that may contain one or more unit dosage forms containing the active ingredient. The pack may, for example, comprise metal or plastic foil, such as a blister pack. The pack or dispenser device is suitably accompanied by instructions for administration.

For topical application, suitable nonsprayable viscous, semi-solid or solid forms can be employed which include a carrier compatible with topical application and having a dynamic viscosity preferably greater than water, for example, mineral oil, almond oil, self-emulsifying beeswax, vegetable oil, white soft paraffin, and propylene glycol. Suitable formulations include, but are not limited to, creams, jellies, gels, pastes, ointments, lotions, solutions, suspensions, emulsions, powders, liniments, salves, aerosols, transdermal patches, etc., which are, if desired, sterilized or mixed with auxiliary agents, e.g., preservatives, stabilizers, demulsifiers, wetting agents, etc. A cream preparation in accordance with the present invention suitably includes, for example, mixture of water, almond oil, mineral oil and self-emulsifying beeswax; an ointment preparation suitably includes, for example, almond oil and white soft paraffin; and a lotion preparation suitably includes, for example, dry propylene glycol. For purposes of transdermal administration, dilute sterile, aqueous or partially aqueous solutions (usually in about 0.1% to 5% concentration), otherwise similar to the above parenteral solutions, are prepared.

Those of ordinary skill in the art will readily optimize effective doses and co-administration regimens (as described hereinbelow) as determined by good medical practice and the clinical condition of the individual patient. Regardless of the manner of administration, it will be appreciated that the actual preferred amounts of active compound in a specific case will vary according to the efficacy of the specific compound employed, the particular compositions formulated, the mode of application, and the particular situs and organism being treated. For example, the specific dose for a particular patient depends on age, sex, body weight, general state of health, on diet, on the timing and mode of administration, on the rate of excretion, and on medicaments used in combination and the severity of the particular disorder to which the therapy is applied. Dosages for a given patient can be determined using conventional considerations, e.g., by customary comparison of the differential activities of the subject compounds and of a known agent, such as by means of an appropriate conventional pharmacological protocol. A physician of ordinary skill can readily determine and prescribe the effective amount of the drug required to counter or arrest the progress of the condition. Optimal precision in achieving concentrations of drug within the range that yields efficacy without toxicity requires a regimen based on the kinetics of the drug's availability to target sites. This involves a consideration of the distribution, equilibrium, and elimination of a drug. The dosage of active ingredient in the compositions of this invention may be varied; however, it is necessary that the amount of the active ingredient be such that an efficacious dosage is obtained. The active ingredient is administered to patients (animal and human) in need of treatment in dosages that will provide optimal pharmaceutical efficacy.

Also included within the scope of the present invention is the co-administration of effective dosages of the analogs of formulas (I)-(IV) in conjunction with hormones or other therapeutic agents, e.g., estrogens, which are known to ameliorate bone diseases or disorders typically associated with hyperparathyroidism and ARVDD syndrome. Such bone agents may include other vitamin D compounds, conjugated estrogens or their equivalents, calcitonin, bisphosphonates, calcium supplements, cobalamin, pertussis toxin and boron.

The term “co-administration” is meant to refer to a combination therapy by any administration route in which two or more agents are administered to a patient or subject. Co-administration of agents may be referred to as combination therapy or combination treatment. The agents may be in the same dosage formulations or separate formulations. For combination treatment with more than one active agent, where the active agents are in separate dosage formulations, the active agents can be administered concurrently, or they each can be administered at separately staggered times. The agents may be administered simultaneously or sequentially (i.e., one agent may directly follow administration of the other or the agents may be give episodically, i.e., one can be given at one time followed by the other at a later time, e.g., within a week), as along as they are given in a manner sufficient to allow both agents to achieve effective concentrations in the body. The agents may also be administered by different routes, e.g., one agent may be administered intravenously while a second agent is administered intramuscularly, intravenously or orally. In other words, the co-administration of the active vitamin D compound in accordance with the present invention with another therapeutic agent is suitably considered a combined pharmaceutical preparation which contains an active vitamin D compound and, e.g., a bone agent, the preparation being adapted for the administration of the active vitamin D compound on a daily or intermittent basis, and the administration of, e.g., a bone agent on a daily or intermittent basis. The agents also may be formulated as an admixture, as, for example, in a single tablet.

Possible dose ranges for exemplary co-administered agents are provided in Table 1.

TABLE 1
Possible Oral Dose Ranges for Various Agents Co-Administered
With Active Vitamin D Compounds of Formulas (I)-(IV)

| | Dose Ranges | | |
|---|---|---|---|
| Agent | Broad | Preferred | Most Preferred |
| Conjugated Estrogens or | 0.3-5.0 | 0.4-2.4 | 0.6-1.2 |
| Equivalent (mg/day) | | | |
| Sodium Fluoride (mg/day) | 5-150 | 30-75 | 40-60 |
| Calcitonin (IU/day) | 5-800 | 25-500 | 50-200 |
| Bisphosphonates (mg/day) | 50-2000 | 100-1500 | 250-1000 |
| Calcium Supplements | 250-2500 | 500-1500 | 750-1000 |
| (mg/day) | | | |
| Cobalamin (μg/day) | 5-200 | 20-100 | 30-50 |
| Pertussis Toxin (mg/day) | 0.1-2000 | 10-1500 | 100-1000 |
| Boron (mg/day) | 0.10-3000 | 1-250 | 2-100 |

Although the above dosages are for oral administration, it is understood that the co-administered agents can also be administered in alternative fashions, including intranasally, transdermally, intrarectally, intravaginally, subcutaneously, intravenously, and intramuscularly. It is also contemplated that some of the co-administered agents may be given on an other than daily basis.

For convenience, the active vitamin D compound in accordance with the present invention and the co-administered therapeutic agent may be packaged together, e.g., in a blister pack or dispenser device. In other words, the active vitamin D compound and the other therapeutic agent may be contained in a common package, each contained in a separate container therein, and also having instructions for use of the compound and agent in the treatment of hyperparathyroidism, e.g., instructions for administering the active vitamin D compound and the therapeutic agent to a subject having hyperparathyroidism and/or suffering from ARVDD on a daily or episodic basis.

Bulk quantities of the vitamin D analogs in accordance with the present invention can be readily obtained in accordance with the many widely known processes, e.g., as described in U.S. Pat. Nos. 3,907,843; 4,195,027; 4,202,829; 4,234,495; 4,260,549; 4,555,364; 4,554,106; 4,670,190; and 5,488,120; WO 94/05630, and Strugnell et al., 310 Biochem. J. 233-241 (1995), all of which are herein fully incorporated by reference.

The present invention is further explained by the following examples, which should not be construed by way of limiting the scope of the present invention.

### EXAMPLES

### Comparison of 1α-OH-vitamin D₂ with 1α-OH-vitamin D₃

A comparison of 1α-(OH)D₂ to 1α-(OH)D₃ has been conducted. 1α-(OH) D₂ is equally active as 1α-(OH)D₃ in the healing of rickets, in the stimulation of intestinal calcium absorption and in the elevation of serum inorganic phosphorous of rachitic rats. [G. Sjoden et al., J. Nutr. 114, 2043-2946 (1984)]. In the same laboratory animal, 1α-OH-D₂ was found to be 5 to 15 times less toxic than 1α-OH-D₃ [see, also, G. Sjoden et al., Proc. Soc. Exp. Biol. Med. 178, 432-436 (1985)]. It has also now been found that, for example, 1α-OH-D₂ may be safely administered for up to two years to human subjects experiencing or having a tendency toward loss of bone mass or bone mineral content at dosages greater than 3 μg/day.

The following examples demonstrate that 1α-(OH)D₂ and 1α,24-(OH)₂D₄ are effective in reducing or preventing elevated blood PTH levels as well as preventing or restoring the loss of bone mass or bone mineral content while being substantially less toxic than 1α,25-(OH)₂D₃ and 1α-(OH)D₃. It is to be understood that although the following examples detail the use of 1α-(OH)D₂ and 1α,24-(OH)D₂D₄, 1α,24-(S)—(OH)₂D₂ may be readily utilized in the treatment of this invention with essentially equivalent results. For example, 1α,24(S)—(OH)₂D₂ shows activity equivalent to 1α,24(R)—(OH)₂D₃ and is also significantly less toxic than its vitamin D₃ counterpart.

### Example 1

### Study Demonstrating Better Safety

The low toxicity of 1α-(OH)D₂ in human patients was demonstrated in a clinical study involving 15 postmenopausal osteoporotic women. [J. Bone Min. Res.; 9:607-614 (1994).] The selected patients were between 55 and 75 years of age, and exhibited L2-L3 vertebral bone mineral density (“BMD”) between 0.7 and 1.05 g/cm², as determined by measurements with a LUNAR dual-photon absorptiometer. (The mean bone mineral density in women with osteoporosis is about 0.85±0.17 g/cm², so that these limits correspond to about the 15th to 85th percentiles.)

On admission to the study, all patients received instruction on selecting a daily diet containing 400 to 600 mg of calcium. Compliance to this diet was verified at weekly intervals by 24-hour food records and by interviews with each patient.

All patients completed a one-week baseline period, a five- to seven-week treatment period, and a one-week post-treatment observation period. During the treatment period, patients orally self-administered 1α-(OH)D₂ at an initial dose of 0.5 μg/day for the first week, and at successively higher doses of 1.0, 2.0, 4.0, 5.0, 8.0 and 10.0 μg/day in each of the following weeks. All doses were administered before breakfast.

Blood and urine chemistries were monitored on a weekly basis throughout the study. Key blood chemistries included fasting serum levels of calcium, phosphorus, osteocalcin, creatinine and blood urea nitrogen. Key urine chemistries included 24-hour excretion of calcium, phosphorus and creatinine.

Data from the study clearly demonstrated that 1α-(OH)D₂ can be safely administered at high dose levels on a daily dosing regimen for periods of several weeks. In particular, the compound did not adversely affect kidney function, as determined by creatinine clearance and blood levels of urea nitrogen; nor did it increase urinary excretion of hydroxyproline, indicating the absence of any stimulatory effect on bone resorption. The compound had no effect on any routinely monitored serum chemistries, indicating the absence of adverse metabolic effects.

A positive effect of 1α-(OH)D₂ on calcium homeostasis was evident from dose-related increases observed in 24-hour urinary calcium levels, confirming that the compound increases intestinal calcium absorption, and from dose-related increases in serum osteocalcin, suggesting that the compound directly stimulates bone formation.

### Example 2

### Study Demonstrating Safety and Efficacy for Human Osteoporosis

The safety and efficacy of 1α-(OH)D₂ as an oral treatment for osteoporosis was confirmed in a study involving 60 postmenopausal osteoporotic outpatients. The selected subjects had ages between 60 and 70 years, and exhibited L2-L3 vertebral BMD between 0.7 and 1.05 g/cm², as determined by dual-energy x-ray absorptiometry (DEXA). Exclusion criteria encompassed significant medical disorders and recent use of medications known to affect bone or calcium metabolism.

On admission to the study, each subject was assigned at random to one of two treatment groups; one group received up to a 104-week course of therapy with 1α-(OH)D₂; the other received only placebo therapy. All subjects received instruction on selecting a daily diet containing 700-900 mg of calcium and were advised to adhere to this diet over the course of the study. Compliance to the diet was verified at regular intervals by 24-hour food records and by interviews with each subject.

During the treatment period, subjects from one group orally self-administered 1α-(OH)D₂ at an initial dosage of 1.0 μg/day for one week, and increased the dosage to 2.0, 3.0, 4.0 μg/day in each of the following weeks, to a maximum dosage of 5.0 μg/day. The dosage for any given subject was increased in this way until the rate of urinary calcium excretion was elevated to approximately 275-300 mg/24 hours, at which point the subject held the dosage constant at the highest level attained. Subjects from the second group self-administered a matching placebo medication every day, titrating the apparent dosage upwards in the same manner as subjects being treated with 1α-(OH)D₂.

Spinal and femoral neck BMD were measured in all subjects by DEXA at the beginning of the study, and at six-month intervals thereafter. Intestinal calcium absorption was estimated in all subjects by a single isotope technique at the beginning of the study, and at 12-month intervals. Serum levels of vitamin D metabolites were determined by radioreceptor binding assays at baseline and at six-month intervals. Serum osteocalcin, serum PTH and urine hydroxyproline also were determined at baseline and at six-month intervals.

Other blood and urine chemistries were monitored at regular intervals during the treatment period. These chemistries included serum calcium, serum ionized calcium, urine calcium, blood urea nitrogen, serum creatinine and creatinine clearance. Kidney-ureter-bladder (KUB) x-rays were obtained at baseline and at 12-month intervals thereafter.

The results of the study are summarized below:

Subjects: Sixty subjects enrolled in what was originally intended to be a 52-week study. Of these 60 subjects, 55 completed one year of treatment (28 active; 27 placebo); and 41 subjects completed an optional second year of treatment.

Test Drug Dosages: The average prescribed dosage for subjects who received 1α-(OH)D₂ was 4.2 μg/day at 52 weeks and 3.6 μg/day at 104 weeks. The average prescribed dosage for placebo subjects was an apparent 4.8 μg/day at 52 weeks and 4.8 μg/day at 104 weeks.

Exclusions: One subject failed to comply with the prescribed dosage of test drug, as confirmed by an absence of serum 1α,25-(OH)₂D₂ at any time during the study. Data for this subject were excluded from analysis. Three patients were diagnosed with hyperparathyroidism when the PTH assays were completed (in batch) at the study's conclusion; data for these subjects were excluded from analysis. No subjects were excluded from analysis for noncompliance with the required dietary calcium intake of 700-900 mg/day.

Episodes of Hypercalcemia/Hypercalciuria: Marked hypercalcemia (>10.8 mg/dL) occurred in one subject in association with an intercurrent illness. The prescribed dosage of 1α-(OH)D₂ at the time of this episode was 5.0 μg/day. Moderate hypercalcemia (10.4-10.8 mg/dL) occurred in two subjects over the course of the study at prescribed dosages of 5.0 μg/day. Mild hypercalcemia (10.2-10.4 mg/dL) occurred in four subjects in the first year and in two subjects in the second year. Hypercalciuria was observed occasionally over the two-year study in 17 subjects treated with 1α-(OH)D₂.

Serum Calcium/Ionized Calcium: Mean serum calcium was approximately 0.1 to 0.2 mg/dL higher in subjects treated with 1α-(OH)D₂ than in subjects treated with placebo. This difference was significant (P<0.05) only during the second year of treatment. Mean serum ionized calcium was approximately 0.05 to 0.10 mg/dL higher in subjects treated with 1α-(OH)D₂.

Urine Calcium: Mean urine calcium increased during the initial titration period in a dose-response fashion. After titration, mean urine calcium was 50 to 130% higher (P<001) with 1α-(OH)D₂ treatment than with placebo treatment.

Kidney Function: No significant changes were observed with long-term 1α-(OH)D₂ treatment in BUN, serum creatinine or creatinine clearance. KUB x-rays revealed no abnormalities in either treatment group throughout the course of the study.

Bone: Bone mineral density (BMD) in the L2-L4 vertebrae progressively increased with 1α-(OH)D₂ treatment and decreased with placebo treatment over the two-year study. The difference in spinal BMD between the treatment groups became statistically significant (P<0.05) after 24 months of treatment. Similar changes were observed in femoral neck BMD with statistically significant differences observed after 18 months (P<0.001) and 24 months (P<0.05) of treatment.

Calcium Uptake: Intestinal absorption of orally administered ⁴⁵Ca increased by 40% (P<0.001) after 52 weeks of 1α-(OH)D₂ therapy, and by 29% (P<0.5) after 104 weeks of 1α-(OH)D₂ therapy, relative to placebo control.

Vitamin D Metabolites: Treatment with 1α-(OH)D₂ caused progressive increases in mean serum total 1α,25-(OH)₂D₃ from 21% (P<0.05) at six months to 49% (P<0.01) at 24 months relative to placebo therapy. This increase resulted from a dramatic rise in serum 1α,25-(OH)₂D₂ which was partially offset by a 50+% decrease in serum 1α,25-(OH)₂D₃. No treatment related changes were apparent in serum total 25-(OH)D.

Biochemical Parameters:

Serum levels of PTH decreased with 1α-(OH)D₂ therapy by 17% at 52 weeks and by 25% at 1-4 weeks, relative to placebo therapy.

Serum levels of osteocalcin were unchanged with long-term 1α-(OH)D₂ therapy.

Fasting urine hydroxyproline:creatinine ratio tended to decrease with long-term 1α-(OH)D₂ treatment but the observed differences between the 1α-(OH)D₂ and placebo treatment groups were not significantly different.

The results of this study clearly indicated that 1α-(OH)D₂ can be tolerated in higher long-term daily dosages than the commonly used vitamin D₃ analogues. They also showed that 1α-(OH)D₂ is well tolerated in postmenopausal women at long-term dosages in the range of 2.0 to 3.0 μg/day, provided that individuals exhibiting abnormally high urine calcium levels (when not receiving vitamin D therapy) are excluded from treatment. Long-term administration of such high dosages of 1α-(OH)D₂ significantly reduced bone loss at the spine and femoral neck, the most frequent sites of osteoporotic fractures. These positive effects on bone were accompanied by a sustained increase in intestinal calcium absorption and a sustained decrease in serum PTH. They were not accompanied by clear long-term trends in serum osteocalcin and urine hydroxyproline. Taken together, the results of this study demonstrate that 1α-(OH)D₂ is safe and effective in the treatment of postmenopausal or senile osteoporosis.

### Secondary Hyperparathyroidism Studies

### Example 3

### Open Label Study in End Stage Renal Disease Patients Exhibiting Secondary Hyperparathyroidism

Five end stage renal disease patients were enrolled in an open label study. The selected patients had ages between 36 and 72 years and had been on hemodialysis for at least 4 months prior to enrollment. The patients each had an average serum phosphorus in the range of 3.0 to less than or equal to 6.9 mg/dL during the two months prior to enrollment (often controlled by oral calcium as a phosphate binder e.g., calcium carbonate or calcium acetate), and had a history of elevated serum PTH values of greater than 400 pg/mL when not receiving 1α,25-(OH)₂D₃ therapy.

Each patient had been receiving 1α,25-(OH)₂D₃ prior to enrollment, and discontinued the 1α,25-(OH)₂D₃ therapy for eight weeks prior to receiving 1α-(OH)D₂. After 8 weeks, the patients received treatment of 1α-(OH)D₂ at a dosage of 4 μg/day for 6 weeks. Throughout the eight-week washout period and the treatment period, patients were monitored weekly or biweekly for serum intact PTH level and weekly for excessive elevation in serum levels of calcium and phosphorus.

Throughout the washout period and treatment period, patients underwent routine hemodialysis (3 times per week) using a 1.25 mM calcium dialysate. They also ingested significant amounts of calcium as phosphate binders (1-10 g elemental Ca) to keep serum phosphorus levels below 6.9 mg/dL.

Average baseline values were as follows: serum PTH—480±21 pg/mL; serum Ca—8±0.3 mg/mL and serum phosphorus—5.1±0.2 mg/mL. In three patients, serum PTH decreased by 68%, 74% and 87% after two weeks. In the other two patients, serum PTH declined by 33% in one and 3% in the other after four weeks. Overall, serum PTH decreased by 49±17% and 33±9% after two and four weeks of 1α-OH—D₂, respectively, (p<0.05). Serum calcium (mg/dL) was 10.2±0.4 (p<0.05) and 9.8±0.2 (NS) and serum phosphorus (mg/dL) was 5.4±0.5 and 5.5±0.8 at two and four weeks, respectively (NS). A rise in serum PTH from the second to fourth weeks of 1α-(OH)D₂ treatment occurred when 1α-(OH)D₂ was withheld in three patients with serum PTH<130; they developed mild hypercalcemia (serum calcium, 10.3-11.4 mg/dL) that reversed after stopping 1α-(OH)D₂. No other adverse effects occurred. At 4-6 weeks of 1α-(OH)D₂ treatment of 4 μg, thrice weekly, four of five patients were in the target range of serum PTH; serum calcium was 10.0±0.2 mg/dL and serum phosphorus, 5.3±0.2 mg/dL. The patient who failed to respond to six weeks of 1α-(OH)D₂ treatment had a delayed response to both intravenous and oral calcitriol earlier, requiring several months of treatment before serum PTH fell. Serum PTH values in this patient fell by 38% after eight weeks of 1α-(OH)D₂ treatment. These data show that 1α-(OH)D₂ is efficacious and safe for the control of secondary hyperparathyroidism in end stage renal disease patients.

### Example 4

### Double Blind Study of Bone in End Stage Renal Disease Patients

A twelve-month double-blind placebo-controlled clinical trial is conducted with thirty-five men and women with renal disease who are undergoing chronic hemodialysis. All patients enter an eight-week control period during which time they receive a maintenance dose of vitamin D₃ (400 IU/day). After this control period, the patients are randomized into two treatment groups: one group receives a constant dosage of 1α-(OH)D₂ (u.i.d.; a dosage greater than 3.0μg/day) and the other group receives a matching placebo. Both treatment groups receive a maintenance dosage of vitamin D₃, maintain a normal intake of dietary calcium, and refrain from using calcium supplements. Oral calcium phosphate binders are used as necessary to maintain serum levels of phosphorus below 7.0 mg/dL. Efficacy is evaluated by pre- and post-treatment comparisons of the two patient groups with regard to (a) direct measurements of intestinal calcium absorption, (b) total body calcium retention, (c) radial and spinal bone mineral density, and (d) determinations of serum calcium and osteocalcin. Safety is evaluated by regular monitoring of serum calcium.

Analysis of the clinical data show that 1α-(OH)D₂ significantly increases serum osteocalcin levels and intestinal calcium absorption, as determined by direct measurement using a double-isotope technique. Patients treated with 1α-(OH)D₂ show normalized serum calcium levels, stable values for total body calcium, and stable radial and spinal bone densities relative to baseline values. In contrast, patients treated with placebo show frequent hypocalcemia, significant reductions in total body calcium and radial and spinal bone density. An insignificant incidence of hypercalcemia is observed in the treated group.

### Example 5

### Double-blind Study in End Stage Renal Disease (ESRD) Patients Exhibiting Secondary Hyperparathyroidism

Up to 120 ESRD (End Stage Renal Disease) patients undergoing chronic hemodialysis are studied in a multicenter, double-blind, placebo-controlled study. The selected patients reside in two major metropolitan areas within the continental U.S., have ages between 20 and 75 years and have a history of secondary hyperparathyroidism. They have been on hemodialysis for at least four months, have a normal (or near normal) serum albumin, and have controlled serum phosphorus (often by using oral calcium phosphate binders).

On admission to the study, each patient is assigned at random to one of two treatment groups. One of these groups receives two consecutive 12-week courses of therapy with 1α-(OH)D₂; the other receives a 12-week course of therapy with 1α-(OH)D₂ followed, without interruption, by a 12-week course of placebo therapy. Each patient discontinues any 1α,25-(OH)₂D₃ therapy for eight weeks prior to initiating 1α-(OH)D₂ therapy (4 μg/day). Throughout this eight-week washout (or control) period and the two subsequent 12-week treatment periods, patients are monitored weekly for serum calcium and phosphorus. Serum intact PTH is monitored weekly or biweekly, and bone-specific serum markers, serum vitamin D metabolites, serum albumin, blood chemistries, hemoglobin and hematocrit are monitored at selected intervals.

During the study, patients undergo routine hemodialysis (three times per week) using a 1.24 mM calcium dialysate and ingest calcium phosphate binders (such as calcium carbonate or calcium acetate) in an amount sufficient to keep serum phosphate controlled (6.9 mg/dL). Patients who develop persistent mild hypercalcemia or mild hyperphosphatemia during the treatment periods reduce their 1α-(OH)D₂ dosage to 4 μg three times per week (or lower). Patients who develop marked hypercalcemia or marked hyperphosphatemia immediately suspend treatment. Such patients are monitored at twice weekly intervals until the serum calcium or phosphorus is normalized, and resume 1α-(OH)D₂ dosing at a rate which is 4 μg three times per week (or lower).

During the eight-week washout period, the mean serum level of PTH increases progressively and significantly. After initiation of 1α-(OH)D₂ dosing, mean serum PTH decreases significantly to less than 50% of pretreatment levels. Due to this drop in serum PTH, some patients need to reduce their dosage of 1α-(OH)D₂ to 4 μg three times per week (or to even lower levels) to prevent excessive suppression of serum PTH. In such patients, exhibiting excessive suppression of serum PTH, transient mild hypercalcemia is observed, which is corrected by appropriate reductions in 1α-(OH)D₂ dosages.

At the end of the first 12-week treatment period, mean serum PTH is in the desired range of 130 to 240 pg/mL and serum levels of calcium and phosphorus are normal or near normal for end stage renal disease patients. For the placebo group, at the end of the second 12-week treatment period (during which time 1α-(OH)D₂ treatment is suspended and replaced by placebo therapy), mean serum PTH values markedly increase, reaching pretreatment levels. This study demonstrates that: (1) 1α-(OH)D₂ is effective in reducing serum PTH levels, and (2) 1α-(OH)D₂ is safer than currently used therapies, despite its higher dosages and concurrent use of high levels of oral calcium phosphate binder.

### Example 6

### Open Label Study of Elderly Subjects with Elevated Blood PTH from Secondary Hyperparathyroidism

Thirty elderly subjects with secondary hyperparathyroidism are enrolled in an open label study. The selected subjects have ages between 60 and 100 years and have elevated serum PTH levels (greater than the upper limit of young normal range). Subjects also have femoral neck osteopenia (femoral neck bone mineral density of ≦0.70 g/cm²).

Subjects are requested to keep a diet providing approximately 500 mg calcium per day without the use of calcium supplements. For a twelve week treatment period, subjects self-administer orally 2.5 μg/day 1α-(OH)D₂. At regular intervals throughout the treatment period, subjects are monitored for serum PTH levels, serum calcium and phosphorus, and urine calcium and phosphorus levels. Efficacy is evaluated by pre- and post-treatment comparisons of serum PTH levels. Safety is evaluated by serum and urine calcium and phosphorus values.

The administration of 1α-(OH)D₂ is shown to significantly reduce PTH levels with an insignificant incidence of hypercalcemia, hyperphosphatemia, hypercalciuria and hyperphosphaturia.

### Example 7

### Double Blind Study of Open Label Study of Elderly Subjects with Elevated Blood PTH from Secondary Hyperparathyroidism

A twelve month double-blind placebo-controlled clinical trial is conducted with forty subjects with secondary hyperparathyroidism. The selected subjects have ages between 60 and 100 years and have a history of secondary hyperparathyroidism. Subjects also have femoral neck osteopenia (femoral neck bone mineral density of ≦0.70 g/cm²).

All subjects enter a six-week control period after which the subjects are randomized into two treatment groups: one group receives a constant dosage of 15 μg/day 1α,24-(OH)₂D₄ (u.i.d.; a dosage greater than 7.5 μg/day), and the other group receives a matching placebo. Both groups maintain a normal intake of dietary calcium without the use of calcium supplements. Efficacy is evaluated by pre- and post-treatment comparisons of the two patient groups with regard to (a) intact PTH (iPTH); (b) radial, femoral and spinal bone mineral density; and (c) bone-specific urine markers (e.g., pyridinium crosslinks). Safety is evaluated by (a) serum calcium and phosphorus, and (b) urine calcium and phosphorus.

Analysis of the clinical data show that 1α,24-(OH)₂D₄ significantly decreases iPTH and bone specific urine markers. Subjects treated with this compound show normal serum calcium levels and stable radial and spinal bone densities relative to baseline values. In contrast, patients treated with placebo show no reduction in iPTH and bone-specific urine markers. An insignificant incidence of hypercalcemia is observed in the treatment group.

### Secondary and Tertiary Hyperparathyroidism Study

### Example 8

### Open Label Study of Renal Patients with Sufficiently Elevated Blood PTH from Secondary and Tertiary Hyperparathyroidism

Fourteen renal patients enrolled in a clinical trial to study secondary hyperparathyroidism showed baseline iPTH levels greater than 1000 pg/mL (range: 1015-4706 pg/mL). These greatly elevated levels indicated a component of the disease as tertiary (i.e., glandular enlargement but continued presence of vitamin D receptors) to the gland as well as a component secondary to the loss of renal function. The initial dose of 1α-(OH)D₂ (10 μg-3 times/week) was increased (maximum, 20 μg-3 times/week) or decreased as necessary to attain and maintain iPTH in the range of 150-300 pg/mL. After 11-12 weeks of treatment, the iPTH levels of all but two of the patients had decreased to below 1000 pg/mL, and the iPTH levels in nine of the patients had decreased to below 510 pg/mL. There were no episodes of hypercalcemia with the patients during the study.

### Example 9

### Placebo-Controlled Study of Elderly Subjects with Elevated Blood PTH From 1,25(OH)₂D₃ Deficiency Associated With ARVDD Syndrome

Sixty elderly subjects with elevated PTH from 1,25(OH)₂D₃ deficiency associated with ARVDD syndrome are enrolled in a blind placebo-controlled study. The selected subjects have ages between 50 and 80 years and have elevated serum PTH levels (greater than the upper limit of normal range) and depressed serum 1,25(OH)₂D₃ levels (below the lower limit of normal range). Subjects also have femoral neck osteopenia (femoral neck bone mineral density of ≦0.70 g/cm²).

Subjects are requested to keep a diet providing approximately 500 mg of calcium per day and are not to use calcium supplements. For a twelve month treatment period, thirty subjects self-administer orally 20 μg of 1α-(OH)D₂ once per week; the other thirty subjects self-administer placebo capsules once per week. At regular intervals throughout the treatment period, subjects are monitored for femoral bone mineral density; serum PTH levels, calcium, phosphorus and osteocalcin; and urine calcium, phosphorus and hydroxyproline levels. Other safety parameters monitored include blood urea nitrogen, serum creatinine and creatinine clearance. Efficacy is evaluated by pre- and post-treatment comparisons of serum PTH levels and femoral neck bone mineral density. Safety is evaluated by serum and urine calcium and phosphorus values.

The administration of 1α-(OH)D₂ is shown to significantly reduce PTH levels and stabilize or increase femoral neck bone mineral density with an insignificant incidence of hypercalcemia and hyperphosphatemia, and no effect on kidney function parameters.

### Example 10

### Placebo-Controlled Study of Elderly Subjects with Elevated Blood PTH From 1,25(OH)₂D₃ Deficiency Associated With ARVDD Syndrome

Sixty elderly subjects with elevated PTH from 1,25(OH)₂D₃ deficiency associated with ARVDD syndrome are enrolled in a blind placebo-controlled study. The selected subjects have ages between 50 and 80 years and have elevated serum PTH levels (greater than the upper limit of normal range) and depressed serum 1,25(OH)₂D₃ levels (below the lower limit of normal range). Subjects also have femoral neck osteopenia (femoral neck bone mineral density of ≦0.70 g/cm²).

Subjects are requested to keep a diet providing approximately 500 mg of calcium per day and are not to use calcium supplements. For a twelve month treatment period, thirty subjects self-administer orally 100 μg of 1,24(OH)₂D₂ once per week; the other thirty subjects self-administer placebo capsules once per week. At regular intervals throughout the treatment period, subjects are monitored for femoral bone mineral density; serum PTH levels, calcium, phosphorus and osteocalcin; and urine calcium, phosphorus and hydroxyproline levels. Other safety parameters monitored include blood urea nitrogen, serum creatinine and creatinine clearance. Efficacy is evaluated by per- and post-treatment comparisons of serum PTH levels and femoral neck bone mineral density. Safety is evaluated by serum and urine calcium and phosphorus values.

The administration of 1,24(OH)₂D₂ is shown to significantly reduce PTH levels and stabilize or increase femoral neck bone mineral density with an insignificant incidence of hypercalcemia and hyperphosphatemia and no effect on kidney function parameters.

In summary, the present invention provides therapeutic methods for treating hyperparathyroidism associated with aging and/or ARVDD. The present invention also provides a method of treating and preventing one or more of the conditions included within the syndrome of ARVDD, e.g., (1) primary vitamin D deficiency, (2) 1,25-(OH)₂D₃ deficiency, and (3) 1,25-(OH)₂D₃ resistance. The methods are suitable for lowering elevated blood parathyroid hormone levels, or maintaining lowered blood PTH levels in subjects with ARVDD syndrome. The methods include administering an effective amount of an active vitamin D compound utilizing a variety of treatment protocols. The method in accordance with the present invention has significantly less resultant hypercalcemia and hyperphosphatemia.

While the present invention has now been described and exemplified with some specificity, those skilled in the art will appreciate the various modifications, including variations, additions, and omissions that may be made in what has been described. Accordingly, it is intended that these modifications also be encompassed by the present invention and that the scope of the present invention be limited solely by the broadest interpretation that lawfully can be accorded the appended claims.

1. A method of treating hyperparathyroidism associated with aging, comprising administering to a subject suffering therefrom an amount of active vitamin D formula sufficient to lower or maintain lowered blood parathyroid hormone (PTH) levels.

2. A method in accordance with claim 1, wherein the hyperparathyroidism is associated with Aging-Related Vitamin D Deficiency (ARVDD) syndrome.

3. A method in accordance with claim 2, wherein the ARVDD syndrome includes one or more conditions which is (1) primary vitamin D deficiency, (2) 1,25-dihydroxyvitamin D₃ deficiency, and (3) 1,25-dihydroxyvitamin D₃ resistance.

4. A method in accordance with claim 2, wherein the active vitamin D is a hydroxyvitamin D compound of formula (I): 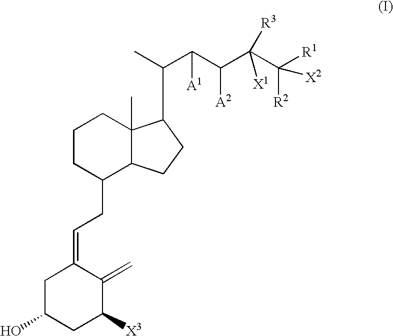
wherein A¹ and A² each are hydrogen or together represent a carbon-carbon bond, thus forming a double bond between C-22 and C-23; R¹ and R² are identical or different and are hydrogen, hydroxyl, lower alkyl, lower fluoroalkyl, O-lower alkyl, lower alkenyl, lower fluoroalkenyl, O-lower alkenyl, O-lower acyl, O-aromatic acyl, lower cycloalkyl with the proviso that both R¹ and R² cannot both be an alkenyl, or taken together with the carbon to which they are bonded, form a C₃-C₈ cyclocarbon ring; R³ is lower alkyl, lower alkenyl, lower fluoroalkyl, lower fluoroalkenyl, O-lower alkyl, O-lower alkenyl, O-lower acyl, O-aromatic acyl or lower cycloalkyl; X¹ is hydrogen or hydroxyl; X² is hydrogen or hydroxyl, or, is taken with R¹ or R², to constitute a double bond; X³ is hydrogen or hydroxyl provided that at least one of X¹, X² and X³ is hydroxyl.

5. A method in accordance with claim 3, wherein the compound of formula (I) is a hypocalcemic hydroxyvitamin D compound.

6. A method in accordance with claim 2, wherein the active vitamin D is a 1α-hydroxyvitamin D compound of formula (II): 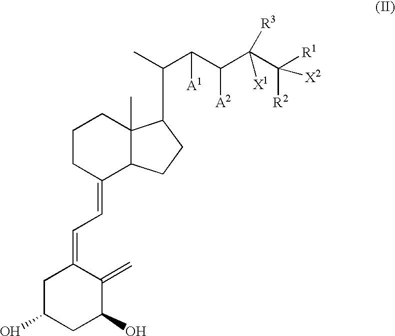
wherein A¹ and A² each are hydrogen or together represent a carbon-carbon bond, thus forming a double bond between C-22 and C-23; R¹ and R² are identical or different and are hydrogen, hydroxyl, lower alkyl, lower fluoroalkyl, O-lower alkyl, lower alkenyl, lower fluoroalkenyl, O-lower alkenyl, O-lower acyl, O-aromatic acyl, lower cycloalkyl with the proviso that both R¹ and R² cannot both be an alkenyl, or taken together with the carbon to which they are bonded, form a C₃-C₈ cyclocarbon ring; R³ is lower alkyl, lower alkenyl, lower fluoroalkyl, lower fluoroalkenyl, O-lower alkyl, O-lower alkenyl, O-lower acyl, O-aromatic acyl or lower cycloalkyl; X¹ is hydrogen or hydroxyl, and X² is hydrogen or hydroxyl, or, is taken with R¹ or R²,to constitute a double bond.

7. A method in accordance with claim 2, wherein the active vitamin D is a 24-hydroxyvitamin D compound of formula (IV): 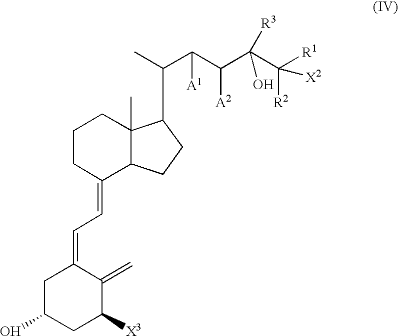
wherein A¹ and A² each are hydrogen or together represent a carbon-carbon bond, thus forming a double bond between C-22 and C-23; R¹ and R² are identical or different and are hydrogen, hydroxyl, lower alkyl, lower fluoroalkyl, O-lower alkyl, lower alkenyl, lower fluoroalkenyl, O-lower alkenyl, O-lower acyl, O-aromatic acyl, lower cycloalkyl with the proviso that both R¹ and R² cannot both be an alkenyl, or taken together with the carbon to which they are bonded, form a C₃-C₈ cyclocarbon ring; R³ is lower alkyl, lower alkenyl, lower fluoroalkyl, lower fluoroalkenyl, O-lower alkyl, O-lower alkenyl, O-lower acyl, O-aromatic acyl or lower cycloalkyl; X³ is hydrogen or hydroxyl; X² is hydrogen or hydroxyl; or, is taken with R¹ or R², to constitute a double bond.

8. A method in accordance with claim 2 wherein the active vitamin D is 1α-hydroxyvitamin D₄; 1α,25-dihydroxyvitamin D₂; 1α-hydroxyvitamin D₂, 1α,24-dihydroxyvitamin D₂; 1α,24,25-trihydroxyvitamin D₂; 1α,25-dihydroxyvitamin D₃, 1α-dihydroxyvitamin D₃; 1α,25-dihydroxyvitamin D₄; 1α,24,25-trihydroxyvitamin D₄; 24-hydroxyvitamin D₂; or 24-hydroxyvitamin D₄.

9. A method in accordance with claim 3 wherein the condition included with ARVDD syndrome is primary vitamin D deficiency.

10. A method in accordance with claim 3 wherein the condition included within ARVDD syndrome is 1,25-dihydroxyvitamin D₃ deficiency.

11. A method in accordance with claim 3 wherein the condition included within ARVDD syndrome is 1,25-dihydroxyvitamin D₃ resistance.

12. A method in accordance with claim 2 wherein the active vitamin D compound is co-administered with at least one agent characterized by said agent's ability to reduce loss of bone mass, or bone mineral content in patients.

13. A method in accordance with claim 12 wherein the agent is other vitamin D compounds, conjugated estrogens, sodium fluorides, bisphosphonates, cobalamin, pertussin toxin or boron.

14. A method in accordance with claim 12 wherein the vitamin D compound is administered before, after or concurrently with the other agent.

15. A method in accordance with claim 2 wherein the active vitamin D is administered in high dose on an intermittent or episodic dosing regime.

16. A method in accordance with claim 15 wherein the amount of active vitamin D is a high dose which is between about 10 μg to about 300 μg.

17. A method in accordance with claim 16 wherein the active vitamin D compound is 1α,25-dihydroxyvitamin D₃ or 1α-dihydroxyvitamin D₃.

18. A method in accordance with claim 15 wherein the high dose is administered once per week to once every 12 weeks.

19. A method in accordance with claim 2 wherein the amount of the vitamin D compound is administered parenterally or orally in combination with a pharmaceutically acceptable carrier.

20. A method in accordance with claim 19 wherein the amount of vitamin D compound is administered parenterally.

21. A method in accordance with claim 20 wherein the amount of vitamin D compound is administered intravenously.

22. A method in accordance with claim 19 wherein the active vitamin D compound is administered orally.

23. A method in accordance with claim 20 wherein the active vitamin D compound is administered in an episodic dose of about 1 μg to about 300 μg.

24. A method in accordance with claim 23 wherein the active vitamin D compound is administered in an episodic dose of about 20 μg to about 100 μg.

25. A method in accordance with claim 19 wherein the active vitamin D compound is co-administered with a phosphate binder.

26. The method of claim 20 wherein the active vitamin D compound is administered is by intravenous injection, nasopharyngeal or mucosal absorption, or transdermal absorption.

27. A method of treating one or more conditions included within aging-related vitamin D deficiency (ARVDD) syndrome in a subject suffering therefrom comprising, administering to the subject an amount of active vitamin D compound sufficient to lower elevated or maintain lowered serum parathyroid hormone levels.

28. A method in accordance with claim 27 wherein the active vitamin D compound is co-administered with at least one agent characterized by the agent's ability to reduce loss of bone mass or bone mineral content in patients experiencing or tending toward the loss of bone mass or bone mineral content.

29. A serum parathyroid hormone level lowering composition, in unit dosage form, comprising an effective amount of a compound of formula (III): 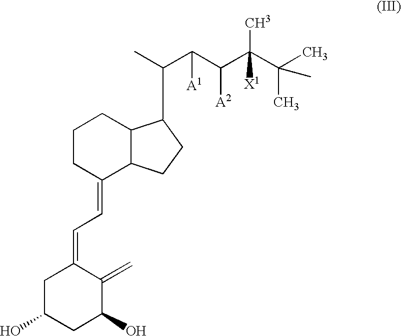
wherein A¹ and A² are either hydrogen or together represent a carbon-carbon double bond between C-22 and C-23; and X¹ is hydrogen or hydroxyl, said effective amount comprising about 1 μg to about 300 μg in unit dosage form, and a pharmaceutically acceptable vehicle for the compound, wherein the amount is effective to lower elevated or maintain lowered serum parathyroid hormone levels of a human in need thereof, following administration thereto.

30. The composition claimed in claim 29 which further comprises, at least one co-administerable agent characterized by the agent's ability to reduce loss of bone mass or bone mineral content in mammals experiencing or tending toward the loss of bone mass or bone mineral content.

31. The composition as claimed in claim 30, wherein the agent is conjugated estrogens, calcitonin, sodium fluorides, bisphosphonates, calcium supplements, cobalamin, pertussin toxin or boron.

32. A co-administerable pharmaceutical combination which comprises the composition of claim 29, and a phosphate binder.

33. The combination of claim 32 wherein said phosphate binder is calcium carbonate or calcium acetate.

34. A method of ameliorating or preventing hyperparathyroidism associated with ARVDD in a subject suffering therefrom, comprising administering to the subject an amount of an active vitamin D compound sufficient to suppress elevated parathyroid activity.

35. A co-administrable pharmaceutical combination comprising (i) a hypocalcemic vitamin D compound and (ii) a bone agent which is conjugated estrogens, calcitonin, sodium fluorides, bisphosphonates, calcium supplements, cobalamin, pertussin toxin or boron.

36. A combination in accordance with claim 35 wherein the vitamin D compound is administered before, after or concurrently with the bone agent.

37. Method of treating 1,25-(OH)₂ D₃ resistance in a subject suffering therefrom comprising, administering to the subject an effective amount of an active vitamin D compound wherein the active vitamin D is administered in high dose on an intermittent dosing regime.

38. A combined pharmaceutical preparation comprising an active vitamin D compound and a bone agent, the preparation being adapted for the administration of the active vitamin D on an episodic basis, and the administration of the bone agent on a daily or episodic basis, to a subject having hyperparathyroidism and/or ARVDD.

39. A pharmaceutical packaging, comprising (i) a plurality of containers therein, at least one of the containers containing an active vitamin D compound, and at least one of the containers containing a bone agent, and (ii) instructions for co-administering the active vitamin D compound and the bone agent to a subject having hyperparathyroidism and/or ARVDD.
